(19) United States
(12) Patent Application Publication
(10) Pub. No.: US 2026/0000601 A1
(43) Pub. Date: Jan. 1, 2026

(54) Anti-Carbonylation Composition And Method Of Protecting Keratin Materials From Carbonylation Stimuli By Using Compositions Including Polylysines

(71) Applicant: ELC Management LLC, Melville, NY (US)

(72) Inventors: Zhen LI, Shanghai (CN); Yuyao LIAO, Shanghai (CN); Xi YANG, Shanghai (CN); Huanjun ZHOU, Shanghai (CN)

(21) Appl. No.: 19/100,524

(22) PCT Filed: Aug. 4, 2022
(86) PCT No.: PCT/CN2022/110203

§ 371 Date: Jan. 31, 2025

(51) Int. Cl. A61K 8/88; A61Q 19/08

(52) U.S. Cl. CPC A61K 8/88; A61Q 19/08

(57) ABSTRACT

The present application relates to a composition of reducing and/or inhibiting protein carbonylation including polylysines. The present application further relates to a method of protecting skin from carbonylation stimuli by using a composition including polylysines, and relates to a non-therapy cosmetic method for caring for keratin materials, especially the skin, by applying the composition of reducing and/or inhibiting protein carbonylation including polylysines.

### TECHNICAL FIELD

The present application relates to an anti-carbonylation composition including polylysine(s), and further relates to a method of reducing and/or inhibiting protein carbonylation, particularly a method of protecting the tissues (for example, keratin materials, especially keratin fiber(s), such as skin etc.) from carbonylation stimuli, by using a composition including polylysine(s).

### BACKGROUND ART

The skin is the body's first barrier against external environmental stimuli. The important mechanism of environmental pollutants such as particulate matter, ultraviolet rays, ozone and the like invading the skin is to attack the unsaturated lipids in the surface layer of the skin through oxidative damage, destroy the outermost protective layer of the skin, and produce active carbonyl substances, such as malondialdehyde, 4-Hydroxy nonenal, acrolein, etc. When the contents of these types of substances exceed the body's scavenging ability, carbonyl stimuli would occur, which induces the carbonylation modification of biological macromolecules such as proteins, resulting in structural changes and losing of functions.

During the protein carbonylation, the reactive aldehydes or ketones are introduced into proteins by oxidation. Protein carbonyls have been shown to be major products of protein oxidation, and may be formed via oxidative cleavage of proteins, direct oxidation of amino acid residues or covalent reaction with aldehydes derived from lipid peroxidation.

It is generally understood that protein carbonylation refers to the process in which the amino or imino groups in the side chain of amino acid residues are attacked by oxygen free radicals and finally converted into aldehyde groups and release NH3+, wherein the bond of the peptide is broken, and a carbonyl group is generated at the break, and the content of the carbonyl group is greatly increased after the amino acid of the protein side chain is attacked by hydroxyl free radicals and modified by oxidation.

Particularly, protein carbonylation occurs because of the direct metal-catalyzed oxidation of amino acid side chains (primary protein carbonylation) or the addition of reactive aldehydes to amino acid side chains (secondary protein carbonylation). As such, reactive aldehydes of exogenous origin, such as acrolein, are well known to be derived from cigarette smoke.

It has been previously reported that primary protein carbonylation plays a role in the mechanism of reactive oxygen species (ROS) signaling. The reactive oxygen species (ROS) in the skin can change the proteins in the cell structure of the skin. If the reactive oxygen species (ROS) cannot be fully controlled by the skin antioxidant defense system, the protein in the skin will be carbonylated, resulting in skin damage.

It has been well documented that the production of carbonyl protein is a hallmark of carbonylation stress, e.g., as an extrinsic factor, such as that induced by ultraviolet radiation or an external application of oxidative chemicals, or as intrinsic factors, such as chemical attack by reactive carbonyls derived from the degradation of lipid peroxides.

Carbonylated proteins in the skin mainly exist in the epidermis and dermis, and the content of carbonylated proteins in the epidermis is higher than that in the dermis. This is because the epidermis of the skin is continuously exposed to the oxidative environment and the unsaturated lipid environment secreted by the sebaceous glands, creating favorable conditions for the continuous generation of reactive aldehyde compounds.

Furthermore, carbonyl stress causes skin damage, such as accelerated skin aging, or various pathological reactions of the skin. Protein carbonylation damages the skin's water-holding capacity, affects the skin's light transmittance, and changes the skin's optical properties etc. Specifically, the presence of carbonylated proteins is associated with changes in skin characteristics: such as mechanical properties; water-holding capacity, including decreased moisture content and increased epidermal water loss; dull skin; and decreased skin clarity. At the same time, increased levels of carbonylated proteins can lead to inflammatory skin diseases such as psoriasis and dermatitis.

Accordingly, there is a great need for anti-carbonylation agents capable of inhibiting or preventing protein carbonylation, as well as skin care compositions and methods comprising anti-carbonylation agents to improve skin appearance or improve at least one sign of skin aging (such as decreased water-holding capacity, dull skin, skin yellowness, decreased skin clarity, elasticity, contrast, etc.) and reduces the occurrence of inflammatory skin diseases such as psoriasis and dermatitis.

The present application is directed to cosmetic compositions comprising at least one polylysine. This present application is also directed to methods for reducing or inhibiting protein carbonylation on keratin materials, especially the skin.

### DETAILED DESCRIPTION

Throughout the description, including the claims, the term “comprising a” should be understood as being synonymous with “comprising at least one”, unless otherwise mentioned. Moreover, the expression “at least one” used in the present description is equivalent to the expression “one or more”.

By “topical application”, it meant that the composition is applied or spread onto the surface of the keratin materials, such as at least one zone of the skin.

As used herein, “anti-carbonylation” means reducing and/or inhibiting protein carbonylation, particularly protecting the tissues (for example, keratin materials, especially keratin fiber(s), such as skin etc.) from carbonylation stimuli.

As used herein, “polylysine” means lysine-based polymer(s).

It has been unexpectedly discovered that the present anti-carbonylation composition (or agent) comprising (or consisting of) at least one polylysine can achieve improved anti-carbonylation efficacy. The present application is directed to the present anti-carbonylation composition (or agent) comprising (or consisting of) at least one polylysine as cosmetic compositions. This present application is also directed to methods for reducing and/or inhibiting protein carbonylation on keratin materials, especially the keratin fiber(s), such as skin, by applying the compositions comprising the present anti-carbonylation composition (or agent) comprising (or consisting of) at least one polylysine.

In one embodiment, the polylysine preferably is L-lysine based polymers.

In another embodiment, the polylysine may be selected from the group consisting of linear α-polylysine, linear ε-polylysine, hyperbranched polylysine or dendritic polylysine.

In one embodiment, the polylysine preferably is the ε-polylysine and/or dendritic polylysine

In other embodiment, the polylysine has the molecular weight of more than 200, even more than 300, or more than 400, preferably more than 500, or more than 800. Particularly, the polylysine has the molecular weight of less than 200000, even less than 150000, or less than 100000, preferably less than 50000, or less than 20000. For example, the polylysine has the molecular weight of between 200 and 200000, even between 400 and 100000, or between 600 and 50000, or between 800 and 20000, or between 1000 and 15000, preferably between 1000 and 10000. Where the polylysine is α-polylysine, a preferred molecular weight of the α-polylysine is lower than 30000.

In other embodiment, the polylysines are present in the cosmetic composition from 0.0001% to 10% by weight, preferably from 0.001% to 5% by weight, preferably from 0.001% to 2% by weight, relative to the total weight of the composition.

In other embodiment, the present composition further comprises a functional agent, which may be, such as, at least one active agent such as skin care agent(s), selected from the group consisting of moisturising agents, such as protein hydrolysates, and polyols, such as glycerol, glycols, polyethylene glycols, and sugar derivatives; natural extracts; vitamins, such as vitamin A (retinol), vitamin E (tocopherol), vitamin C (ascorbic acid), vitamin B5 (panthenol), vitamin B3 (niacinamide), the derivatives of these vitamins (in particular esters) and their mixtures; urea; caffeine; salicylic acid and its derivatives; alpha-hydroxy acids, such as lactic acid and glycolic acid, and their derivatives; retinoids, such as carotenoid and vitamin A derivatives; sunscreen; essential oils of mint, aloe vera or ginseng and their mixtures.

Another subject of the present application is to provide a process of preparing the anti-carbonylation composition, comprising the following steps:
(1) optionally, providing a formula base and then cooled, for example, cooled to room temperature,
(2) dispersing the obtained polylysine, for example, in water and
(3) mixing 1) formula base, and 2) polylysine(s) solution at room temperature.

Another subject of the present application is to provide a non-therapy cosmetic method for countering denaturation of keratin materials, especially the skin, by applying the present composition.

Another subject of the present application is to provide a non-therapy cosmetic method for caring for keratin materials, especially the skin, by applying the present anti-carbonylation composition.

Another subject of the present application relates to a use of the present composition or preparing a product of caring for keratin materials, especially the skin.

Another subject of the present application is to provide a cosmetic product, comprising the present composition.

Other characteristics and advantages of the present application will emerge more clearly on reading the description and the examples that follow.

In the application, unless specifically mentioned otherwise, contents, parts and percentages are expressed on a weight basis.

### Polylysines

Polylysines refer to several types of lysine-based homopolymers, which may differ from each other in terms of stereochemistry and link position. Lysine (an essential amino acid for humans) is the primary building block of polylysine, and is available in two chiral forms: L-lysine and D-lysine. Polymerization of specific chiral lysine monomer will result in L-polylysine and D-polylysine respectively. The precursor amino acid lysine contains two amino groups, one at the α-carbon and one at the ε-carbon. Either can be the location of polymerization, resulting in α-polylysine or ε-polylysine. Polylysine can further be classified according the topology of the polymer chain into linear, hyperbranched and dendritic polylysine. The molecular weight of polylysine can range from 100-1000000.

Polylysines, in this application, refers to lysine-based polymers, such as linear ε-polylysine (ε-PL), linear α-polylysine (α-PL), hyperbranched polylysine and dendritic polylysines.

Typically, polylysines are present in the topical composition at the following concentrations: 0.0001% to 10% by weight, preferably from 0.001% to 5% by weight, preferably from 0.001% to 2% by weight, relative to the total weight of the composition.

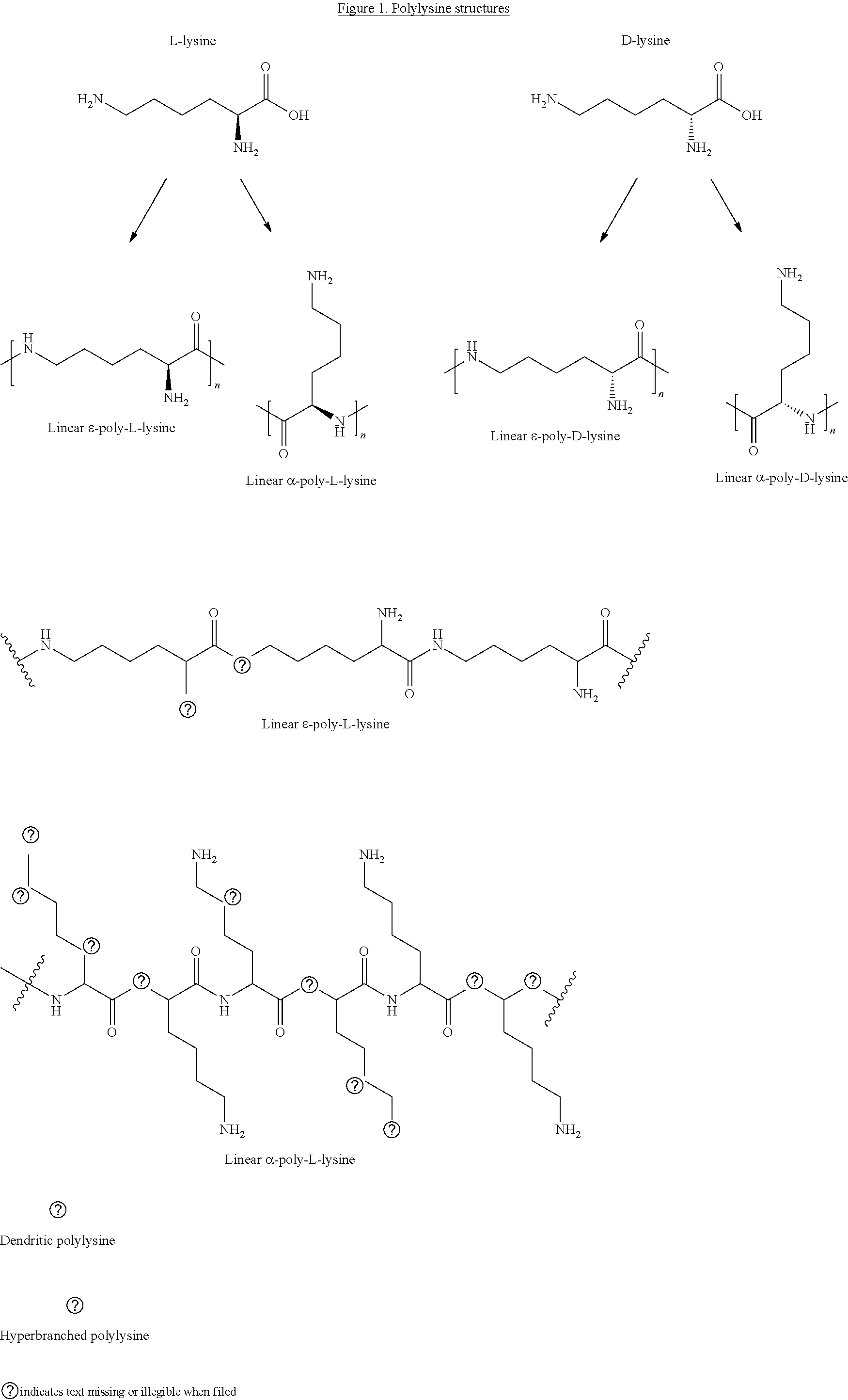

The applicant surprisingly found that the polylysine outperformed monomeric lysine in anti-carbonylation efficacy, optionally with its concentration, for example, ranging from 0.005% to 0.05%, without wishing to be bound by theory, which could possibly contribute to the “multivalent effect” of the polymeric chain.

Surprisingly, without wishing to be bound by theory, the polylysine is preferably selected from the ε-polylysine and/or dendritic polylysine.

### Active Agents

According to an embodiment of the present application, the products of the present application may comprise at least one functional agent, such as, at least one active agent, for example, selected from the group consisting of: desquamating or moisturizing agents; depigmenting or anti-depigmenting agents; anti-glycation agents; anti-NO agents; agents for stimulating the synthesis of dermal or epidermal macromolecules and/or for preventing their degradation; agents for stimulating fibroblast or keratinocyte proliferation and/or keratinocyte differentiation; muscle relaxants or dermo-decontracting agents; free-radical scavengers or anti-pollution agents; tensioning agents; agents acting on the capillary circulation; certainly, the following particularly active agents, and mixtures thereof.

Amongst all of the active agents that can be used in the present application, particular mention can be made of: α- or β-hydroxy acids such as lactic acid, glycolic acid, citric acid, 5-octanoylsalicyclic acid, α-hydroxydecanoic acid, α-hydroxylauric, tartaric acid, glucuronic acid, galacturonic acid, acrylic acid, α-hydroxybutyric acid, α-hydroxyisobutyric acid, malic acid, mandelic acid, phosphoric acid, pyruvic acid, lactobionic acid, and salicylic acid.

It is also possible to use anti-acne agents, such as salicylic acid or benzoyl peroxide, octopirox, dextrorotary and levorotary sulfur-containing amino acids, their salts, and their N-acetyl derivatives such as N-acetyl cysteine, or agents seeking to prevent aging of the skin and/or to improve its state, for example the above-mentioned α- and β-hydroxy acids, retinoids such as retinoic acid, retinol, and its esters, such as, for example, retinyl propionate, and retinyl acetate, or retinyl palmitate, niacinamide, allantoin, extracts of aloe, azelaic acid, bisabolol, phytic acid, collagen, or agents stimulating the formation of collagen, vitamins such as vitamin C or derivatives thereof, such as ascorbyl glucoside, vitamin E or derivatives thereof, vitamin A or derivatives thereof, vitamin F or derivatives thereof, dextrorotary and levorotary sulfur-containing amino acids and derivatives thereof as mentioned above, elastin, N-acetyl D-glucosamine, luteolin, or antioxidants such as green tea or active fractions thereof, glycerin, laponite, caffeine, essential aromatic oils, coloring agents, free-radical scavengers, moisturizers, depigmenting agents, agents for improving the color of the skin such as artificial-tanning agents of the dihydroxyacetone or tyrosin ester type, liporegulators, softeners, anti-wrinkle agents, keratolytic agents, fresheners, deodorants, anesthetics, nourishing agents, and mixtures thereof. It is also possible to use bleaching agents such as kojic acid, ascorbyl phosphates, ascorbyl glucosides, ascorbic acid, and mixtures thereof.

It is also possible to use active agents for improving the condition of the skin in the case of mask, such as moisturizers or agents serving to improve the natural lipid barrier, such as ceramides, cholesterol sulfates, and/or fatty acids, and mixtures thereof. It is also possible to use enzymes that have activity on the skin, such as proteases, lipases, cerebrosidases, and/or melanases, and mixtures thereof.

As other examples of active agents that can be suitable for implementing the present application there are agents for drugs, peptides, proteins, detectable labels, contrast reagents, pain-killing, anesthetics, antibacterial agents, anti-yeast agents, antifungal agents, antiviral agents, anti-dermatitis agents, anti-pruritic agents, anti-emetics, vascular protectors, agents against motion sickness, anti-irritants, anti-inflammatory agents, immunomodulators, anti-hyperkeratolytic agents, agents for treating dry skin, antiperspirants, anti-psoriatic agents, antidandruff agents, anti-aging agents, anti-asthmatic agents and bronchodilators, sunscreen agents, antihistamines, healing agents, corticosteroids, tanning agents, and mixtures thereof.

The content of the at least one active agent in the composition may be adjusted as a function of the intended purpose of the composition.

### Adjuvants

In a known manner, the composition of the present application may also contain adjuvants that are common in cosmetics and/or dermatology, such as preserving agents, antioxidants, pH modifiers (acidic or basic), fragrances, fillers, bactericides, odour absorbers, colorants (pigments and dyes), emulsifiers, and also lipid vesicles.

Needless to say, a person skilled in the art will take care to select this or these optional additional compound(s), and/or the amount thereof, such that the benefits of the composition according to the present application are not, or are not substantially, adversely affected by the envisaged addition.

Notwithstanding that the numerical ranges and parameters setting forth the broad scope of the present application are approximations, the numerical values set forth in the specific examples are reported as precisely as possible. Any numerical value, however, inherently contain certain errors necessarily resulting from the standard deviation found in their respective measurements. The following examples are intended to illustrate the present application without limiting the scope as a result.

### EXAMPLES

The ingredient amounts/concentrations in the compositions/formulas described below are expressed in weight part.

### I. Evaluation of the Present Composition

### 1. In Vitro Anti-Carbonylation Efficacy Test

This assay provides an in vitro method to determine how an anti-carbonylation agent/composition affects the extent of protein carbonylation. With deionized water, the anti-carbonylation agent/composition to be tested was made into a 1% solution, Bovine Serum Albumin (BSA) was made into a 10 mg/mL solution, and Acrolein was made into a 10 mM solution. Then, in a 1.5 mL centrifuge tube, according to the content in Table 1, BSA (10 mg/mL), anti-carbonylation agent/composition (1%), Acrolein (10 mM) and Phosphate buffer solution (PBS) were added to the centrifuge tube. The sample containing only BSA and PBS served as a baseline, and the sample containing BSA, Acrolein and PBS served as a blank. The final liquid volume in each centrifuge tube was 1.0 mL. Each group contained three replicate samples, and all samples were placed at 37° C. overnight.

TABLE 1

| | Anti-carbonylating | | 1% Anti- | 10 mM | |
|---|---|---|---|---|---|
| | agents and working | 10 mg/mL | carbonylating | Acrolein | PBS |
| | concentration | BSA (μL) | agents (μL) | (μL) | (μL) |
| Control | baseline (with no | 100 | 0 | 0 | 900 |
| | active added) | | | | |
| | blank (with no active | 100 | 0 | 100 | 800 |
| | added) | | | | |
| Bench mark | L-Lysine 0.005% | 100 | 5 | 100 | 795 |
| | L-Lysine 0.05% | 100 | 50 | 100 | 750 |
| Linear α- PL | α- PL (1K-5K) | 100 | 5 | 100 | 795 |
| | 0.005% | | | | |
| | α- PL (1K-5K) | 100 | 50 | 100 | 750 |
| | 0.05% | | | | |
| | α- PL (30K-70K) | 100 | 5 | 100 | 795 |
| | 0.005% | | | | |
| | α- PL (30K-70K) | 100 | 50 | 100 | 750 |
| | 0.05% | | | | |
| | α- PL (70K-150K) | 100 | 5 | 100 | 795 |
| | 0.005% | | | | |
| | α- PL (70K-150K) | 100 | 50 | 100 | 750 |
| | 0.05% | | | | |
| Linear ε-PL | ε-PL¹ 0.005% | 100 | 5 | 100 | 795 |
| | ε-PL 0.05% | 100 | 50 | 100 | 750 |
| Dendritic PL | Dendritic PL² | 100 | 5 | 100 | 795 |
| | 0.005% | | | | |
| | Dendritic PL | 100 | 50 | 100 | 750 |
| | 0.05% | | | | |

¹ε-PL is from Chisso corporation
²Dendritic PL is DENDIRCLEAR from Lucas Meyer.

The protein carbonyl content in the above samples was detected by using the Protein Carbonyl Assay Kit of Nanjing Jiancheng Bioengineering Institute. The detection methods are as follows: take 0.1 mL of sample into the assay tube, then add 0.4 mL of reagent 3 in the kit, vortex to mix for 1 min, and react at 37° C. for 30 min in the dark. Add 0.5 mL of reagent 5 in the kit, vortex and mix for 1 min, centrifuge at 12,000 r/min for 10 min at 4° C., discard the supernatant, and leave the pellet. Add 1.0 mL of anhydrous ethanol and ethyl acetate (1:1) mixed solution, vortex to mix for 1 min, centrifuge at 12,000 r/min for 10 min at 4° C., discard the supernatant, leave the precipitate, and repeat the above steps four times. Add 1.25 mL of reagent VI in the kit, mix well, place it in 37° C. for 15 min, vortex to mix, dissolve all the precipitates, and centrifuge at 12,000 r/min for 15 minutes. Take the supernatant in a quartz cuvette with a light diameter of 0.5 cm, adjust the zero with reagent six, and measure the absorbance value of each tube at 370 nm (ultraviolet). According to the formula, we can get the protein carbonyl content.

$$The protein carbonyl content ( nmol / mg prot ) = \frac{Measured OD value - Control OD value}{\begin{matrix}22 \times Colorimetric Path ( cm ) \times \\ Sample protein concentration ( mg prot / L )\end{matrix}} \times 125 \times {10}^{5}$$

The protein content of the samples was determined using Thermos's Micro BCA™ Protein Assay Kit. First, prepare Diluted Albumin (BSA) Standards (Working Range=20-750 μg/mL). Mix BCA Reagent A with Reagent B (Reagent A:Reagent B=50:1) to prepare working solution. Take 25 μL each of the protein standard at different dilution concentration and the protein sample, add them to the microplate. Add 200 μL of working solution to each well and mix well by shaking on a shaker for 30 s. The microplate was sealed and incubated at 37° C. for 30 min. Cool the microplate to room temperature and measure the absorbance of the sample at a wavelength of 562 nm. The corrected absorbance value is the absorbance value of the blank sample subtracted from the readings of each standard and sample. The protein concentration was read against a standard curve prepared with known amounts of BSA. Referring to the standard curve, according to the corrected absorbance value of each protein sample, read the protein concentration of each sample within the linear range of the standard curve. Calculate the amount of protein in the original sample based on the sample volume and dilution.

### 2. Anti-Carbonylation Efficacy of Polylysine

The anti-carbonylation capability of each anti-carbonylation agents/composition can be obtained by dividing the carbonylation degree of the sample by the BSA protein content. The results are shown in the Table 2 and Table 3 below.

TABLE 2

| | | Percentage of | % inhibition | | P value |
|---|---|---|---|---|---|
| | | protein | of | | (vs |
| | Anti-carbonylating | carbonylation | carbonylation | | bench |
| | agents | level (average) | (average) | SD | mark) |
| Control | blank (with no active | 100% | 0% | NA | NA |
| | added) | | | | |
| Bench mark | L-Lysine 0.005% | 79% | 21% | 5% | NA |
| Linear α-PL | α- PL (1K-5K) 0.005% | 50% | 50% | 2% | 0.0021 |
| | α- PL (30K-70K) 0.005% | 89% | 11% | 14% | 0.4163 |
| | α- PL (70K-150K) | 84% | 16% | 9% | 0.5880 |
| | 0.005% | | | | |
| Linear ε-PL | ε-PL 0.005% | 33% | 67% | 3% | 0.0010 |
| Dendritic PL | Dendritic PL 0.005% | 43% | 57% | 3% | 0.0016 |

TABLE 3

| | | Percentage of | % inhibition | | P value |
|---|---|---|---|---|---|
| | | protein | of | | (vs |
| | Anti-carbonylating | carbonylation | carbonylation | | bench |
| | agents | level (average) | (average) | SD | mark) |
| Control | blank (with no active | 100% | 0% | NA | NA |
| | added) | | | | |
| Bench mark | L-Lysine 0.05% | 60% | 40% | 14% | NA |
| Linear α- PL | α- PL (1K-5K) 0.05% | 49% | 51% | 11% | 0.3474 |
| | α- PL (30K-70K) | 78% | 22% | 4% | 0.0928 |
| | 0.05% | | | | |
| | α- PL (70K-150K) | 60% | 40% | 4% | 0.4537 |
| | 0.05% | | | | |
| Linear ε-PL | ε-PL 0.05% | 5% | 95% | 3% | 0.0025 |
| Dendritic PL | Dendritic PL 0.05% | 19% | 81% | 11% | 0.0160 |

The polylysine reduces and/or inhibits the protein carbonylation as shown above.

### II. Formulation Examples

The composition of the invention containing polylysine is prepared by thoroughly mixing these ingredients as follows.

| Composition | 1 | 2 | 3 | 4 |
|---|---|---|---|---|
| Oleth-3 phosphate | 0.45 | 0.45 | 0.45 | 0.45 |
| Oleth-3 | 0.35 | 0.35 | 0.35 | 0.35 |
| Oleth-5 | 0.24 | 0.24 | 0.24 | 0.24 |
| Butylene glycol | 1.3 | 1.3 | 1.3 | 1.3 |
| Squalane | 0.5 | 0.5 | 0.5 | 0.5 |
| BHT | 0.1 | 0.1 | 0.1 | 0.1 |
| Ethylhexyl methoxycinnamate | 0.1 | 0.1 | 0.1 | 0.1 |
| Choleth-24/ceteth-24 | 0.1 | 0.1 | 0.1 | 0.1 |
| Triethanolamine | 0.61 | 0.61 | 0.61 | 0.61 |
| Retinyl palmitate/zea mays | 0.1 | 0.1 | 0.1 | 0.1 |
| (corn) oil/BHT/BHA | | | | |
| Bisabolol | 0.1 | 0.1 | 0.1 | 0.1 |
| Methyl paraben | 0.46 | 0.46 | 0.46 | 0.46 |
| PEG-75 | 4 | 4 | 4 | 4 |
| Bis-PEG-18 methyl ether | 2 | 2 | 2 | 2 |
| dimethyl silane | | | | |
| Glycereth-26 | 1 | 1 | 1 | 1 |
| Methyl gluceth-20 | 4 | 4 | 4 | 4 |
| Trisodium EDTA | 0.1 | 0.1 | 0.1 | 0.1 |
| Pantethine | 0.14 | 0.14 | 0.14 | 0.14 |
| Xanthan gum | 0.075 | 0.075 | 0.075 | 0.075 |
| Phenoxyethanol | 0.72 | 0.72 | 0.72 | 0.72 |
| Sodium ribonucleic acid | 0.01 | 0.01 | 0.01 | 0.01 |
| Sodium hyaluronate | 0.01 | 0.01 | 0.01 | 0.01 |
| ε-Polylysine 25% solution | 0.4 | 0.2 | | |
| (available from Chisso, | | | | |
| Mw = 3200-4500) | | | | |
| DendriClear (Available from | | | 0.4 | 0.2 |
| Lucas Meyer, dendritic | | | | |
| polylysine, Mw = 6171) | | | | |
| Water | To 100 | To 100 | To 100 | To 100 |

### III. Sensory Evaluation of Anti-Carbonylation Compositions

The sensory properties of compositions 1 to 4 were evaluated after applying them to the skin, and are summarized in the table below:

| Composition | Sensory evaluation |
|---|---|
| 1 | good spreading, good residual softness, medium |
| | stickiness |
| 2 | good spreading, good residual softness, low stickiness |
| 3 | good spreading, good residual softness, low stickiness |
| 4 | good spreading, good residual softness, low stickiness |

The cosmetic properties of compositions 1 to 4 were good, especially in terms of spreading, softness and non-stickiness.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. An anti-carbonylation composition comprising at least one polylysine.

2. The anti-carbonylation composition as claimed in claim 1, wherein the polylysine is selected from the group consisting of at least one lysine-based polymer.

3. The anti-carbonylation composition as claimed in claim 1, wherein the at least one polylysine is L-lysine based polymers.

4. The anti-carbonylation composition as claimed in claim 1, wherein the at least one polylysine is selected from the group consisting of α-polylysine, ε-polylysine, hyperbranched polylysine and dendritic polylysine.

5. The anti-carbonylation composition as claimed in claim 1, wherein the at least one polylysine has the molecular weight of more than 200.

6. The anti-carbonylation composition as claimed in claim 1, wherein the polylysine is α-polylysine, and the molecular weight of α-polylysine is lower than 30000.

7. The anti-carbonylation composition as claimed in claim 1, wherein the at least one polylysine is present in the composition from 0.0001% to 10% by weight, relative to the total weight of the composition.

8. The anti-carbonylation composition as claimed in claim 1, wherein the composition further comprises at least one functional agent selected from the group consisting of moisturising agents; urea; caffeine; salicylic acid and its derivatives; alpha-hydroxy acids and their derivatives; retinoids; sunscreen; essential oils of mint, aloe vera or ginseng; and their mixtures.

9. Cosmetic product, comprising the anti-carbonylation composition according to claim 1.

10. Process of preparing the anti-carbonylation composition as claimed in claim 1, comprising the following steps:
(1) providing a formula base and then cooling to room temperature,
(2) dispersing the at least one polylysine in water, to form a polylysine solution,
and
(3) mixing the formula base and the polylysine solution at room temperature.

11. A non-therapeutic cosmetic method for reducing and/or inhibiting protein carbonylation on a keratin material, by applying the anti-carbonylation composition as claimed in claim 1 on the keratin material.

12. A non-therapeutic cosmetic method for caring for a keratin material, by applying the anti-carbonylation composition as claimed in claim 1 on the keratin material.

13-14. (canceled)

15. The anti-carbonylation composition as claimed in claim 5, wherein the at least one polylysine has a molecular weight of less than 200000.

16. The anti-carbonylation composition as claimed in claim 5, wherein the at least one polylysine has a molecular weight between 1000 and 10000.

17. The anti-carbonylation composition as claimed in claim 8, wherein the vitamin is selected from the group consisting of retinol, tocopherol, ascorbic acid, panthenol, niacinamide, a derivative thereof, and a mixture thereof.

18. The anti-carbonylation composition as claimed in claim 8, wherein the moisturising agent is selected from the group consisting of protein hydrolysates, polyols, glycols, and sugar derivatives thereof.

19. The non-therapeutic cosmetic method of claim 11, wherein the keratin material is skin.

20. The non-therapeutic cosmetic method of claim 12, wherein the keratin material is skin.
